(19) United States
(12) Patent Application Publication
(10) Pub. No.: US 2013/0078316 A1
(43) Pub. Date: Mar. 28, 2013

(54) METHODS AND COMPOSITIONS FOR IRRIGATION OF MUCOSAL TISSUES

(71) Applicants: Robert D. Christensen, Layton, UT (US); Edward B. Walker, Ogden, UT (US)

(72) Inventors: Robert D. Christensen, Layton, UT (US); Edward B. Walker, Ogden, UT (US)

(21) Appl. No.: 13/430,594

(22) Filed: Mar. 26, 2012

Related U.S. Application Data
(60) Provisional application No. 61/467,905, filed on Mar. 25, 2011.

(51) Int. Cl. A61K 33/42

(52) U.S. Cl. CPC A61K 33/42
USPC 424/602

(57) ABSTRACT

A synthetic endotracheal irrigant for preventing, treating, and reducing infections, including mucosal infections in patients, including infants. A method for producing a formulation of the synthetic endotracheal irrigant with a water content of about 98% (w/w) to about 99.9(w/w) and a total mineral content of less than about 5.0% (w/w), including a sodium content of less than about 2.0% (w/w). The synthetic endotracheal irrigant excludes protein.

### CROSS-REFERENCE TO RELATED APPLICATIONS

This application claims the benefit of U.S. Provisional Application No. 61/467,905, filed on Mar. 25, 2011, entitled “Methods and Compositions for Irrigation of Mucosal Tissues,” which is incorporated by reference herein in its entirety.

### TECHNICAL FIELD

Embodiments of the present invention relate to methods and compositions for irrigation of body tissues, the treatment of dry mouth, and lubrication of patients who are intubated with an endotracheal tube or receiving some form of mechanical ventilation or other assistance. Embodiments of the invention also relate to methods and formulations for preventing, treating, and reducing infections, including mucosal infections in patients, including infants.

### BACKGROUND

Patients requiring intubation or other ventilation assistance for extended periods often suffer from a high rate of nosocomial infections (See Pediatr Infect Dis J. 1998 July; 17(7):593-8). This tendency is particularly marked among small and ill neonates, patients with immune dysfunction, and patients requiring therapy that reduces innate immunity (such as cancer chemotherapy). Many infections in these groups of patients are caused by organisms of low virulence in healthy adults, such as Candida albicans and Staphylococcus epidermitis. The poor immunologic barrier function in the trachea, nose, and mouth, arising from the presence of an endotracheal tube, a nasal canula, other respiratory support equipment, or subject to nasal continuous positive airway pressure (CPAP) or invasive mechanical assistance, likely contributes to the high incidence of these infections. Normal human saliva, nasal secretions, and tracheal effluent have antimicrobial properties that inhibit colonization of the upper airway by Candida albicans and other infectious agents and protect the upper airway from infection.

Intensive care unit workers and caregivers periodically irrigate the endotracheal tube or other similar lumens of patients who require mechanical ventilation. This treatment is needed to prevent tracheal secretions from building up and occluding the lumen of the endotracheal tube and thereby impeding ventilation and oxygenation. Caregivers also periodically moisten the mucosal tissues of these patients, because the presence of an endotracheal tube prevents lip apposition and therefore results in a dry mouth. Similarly, patients on nasal CPAP or nasal canula oxygen require occasional irrigation of the nose to treat or prevent thick secretions from occluding the nasal passages.

Conventional practices in care facilities involve periodic irrigation with sterile 0.9% (w/w) saline, dispensed from plastic, disposable “bullet tubes” of patients receiving ventilation assistance. These treatments are generally given by the respiratory therapist or the bed side nurse, as often as hourly or as infrequently as every 6 to 8 hours in accordance with local policy and practice, and according to need, as judged by the volume and viscosity of the secretions in the patient's trachea, mouth, and nose.

Salivary and tracheal secretions include an important component called “LL37,” a 37 amino acid peptide (Mol Cell 2:397-403). LL37 also known as human cathelicidin, has widespread antimicrobial properties. LL37 and proteins like it reduce colonization of the airway by pathogenic microorganisms. Research reported by Welsh (AmJ. Respir. Cell Mol. Biol., 1999; 20:872-879), teaches that the electrolyte composition of airway surface liquid (ASL) influences antimicrobial activity of innate host defense and proposes delivery of nonionic osmolytes to draw water to airway surface liquids.

A previous technology presented for periodic airway care and for relieving dry mouth is described in “ETCare: a randomized, controlled, masked trial comparing two solutions for upper airway care in the NICU” (See Journal of Perinatology (2007) 27, 479-484) and “A Low-Sodium Solution for Airway Care: Results of a Multicenter Trial” (See Respir Care 2010; 55(12):1680-1685), which are co-authored by at least one inventor of the present application. The previous technology is also described in International Patent Application No. PCT/US2007/010448 (Intl Pub. No. WO 2006/102438 A2), which was co-invented by at least one inventor of the present application, and is incorporated herein in its entirety. The previous technology includes an aqueous mucosal tissue irrigant and lubricant having less than about 0.3% (w/w) sodium chloride. The irrigant also includes recombinant or purified albumin, as well as alkaline and alkaline earth metal salts, in which the anions of the salts include one or more of chloride, phosphate and sulfate. This conventional formulation exhibit sufficient lubricity to prevent or wash away mucosal buildup causing occlusion in and around respiratory equipment without deactivating the antimicrobial activity of innate host defense.

In general, the previous technology attempts to synthesize human endotracheal fluid, which includes a protein called albumin. Albumin is a protein found in humans and other living organisms. While the inclusion of albumin seemed necessary in the previous technology, in order to most closely synthesize natural endotracheal fluid, it has been discovered the including albumin in synthesized endotracheal fluid results in some difficulties for certain processes of manufacturing and using the synthesized endotracheal fluid.

As one specific example of a problem encountered during the manufacturing process of synthetic endotracheal fluid which includes albumin, it was discovered that thermal sterilization of synthetic endotracheal fluid which includes albumin results in the formation of precipitates. Precipitates are solid particles which separate from a solution or suspension. The formation of precipitates in the synthetic endotracheal fluid gives the appearance of contaminated or “dirty” fluids, which is undesirable in medical applications in which the synthesized endotracheal fluid might be used to flush a patient's mouth or trachea.

As one specific example of a problem encountered during the use of synthetic endotracheal fluid which includes albumin, it was discovered that the albumin causes a bubbles to form in nebulizer and other mechanical deployment applications. These bubbles can obstruct and/or cause undesirable buildup on mechanical parts.

As another potential disadvantage of using synthetic endotracheal fluid which includes albumin, some patients may have concerns about potential allergenic and/or immunological responses to albumin or other proteins. While such concerns may not be based on actual studies or available data, the possibility of such a perception may impact the commercial viability of the previously described synthetic endotracheal fluid which includes albumin.

Another potential problem is the formation of small amounts of precipitate or crystals from compounds or molecules containing calcium or phosphates which may be formed during the preparation stage or after a solution has been stored for a period of time. Even if these deposits may not be dangerous for consumption, their observation may be undesirable for consumer perception of the solution.

### SUMMARY

Embodiments of a synthetic endotracheal irrigant are described for preventing, treating, and reducing infections, including mucosal infections in patients, including infants. A method for producing a formulation of the synthetic endotracheal irrigant with a water content of about 98% (w/w) to about 99.9(w/w) and a total mineral content of less than about 5.0% (w/w), including a sodium content of less than about 2.0% (w/w). The synthetic endotracheal irrigant excludes protein.

In another embodiment, the synthetic endotracheal irrigant substantially mimics natural endotracheal fluids in terms of mineral content and fluid characteristics, but excludes protein. An embodiment of the synthetic endotracheal irrigant consists essentially of: water in a relative quantity of about 95.0% (w/w) to about 99.8% (w/w); potassium phosphate, monobasic, anhydrous in a relative quantity of about 0.001% (w/w) to about 0.04% (w/w); dipotassium phosphate, dibasic, anyhydrous in a relative quantity of about 0.001% (w/w) to about 0.04% (w/w); sodium chloride in a relative quantity of about 0.03% (w/w) to about 0.06% (w/w); potassium chloride in a relative quantity of about 0.10% (w/w) to about 0.30% (w/w); calcium chloride, dihydrate in a relative quantity of about 0.0005% (w/w) to about 0.0300(w/w); and magnesium sulfate, heptahydrate in a relative quantity of about 0.005% (w/w) to about 0.030% (w/w). In this embodiment, the percentages are weight-weight percentages.

Other embodiments of the synthetic endotracheal irrigant are also described.

Other aspects and advantages of embodiments of the present invention will become apparent from the following detailed description, taken in conjunction with the accompanying drawings, illustrated by way of example of the principles of the invention.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment. Also, for reference, all references to % units are actually % (w/w), unless noted otherwise.

Conventional irrigation of mucosal tissue has been carried out with saline solutions of 0.9% (w/w) sodium chloride. Sodium and chloride in saline solutions are common electrolytes found in biological systems. Such systems must maintain electrolytic balance for continued vitality. Electrolytes attract fluids by osmosis. The selection of sodium chloride concentration in solutions applied to tissues has been correlated with the typical physiological sodium chloride concentrations found within cells. When hypertonic saline solutions envelope a cell, osmotic pressure drives water across the cell membrane and outside the cell causing cells to crenate or shrink. When hypotonic saline solutions envelop a cell, osmotic pressure drives water across the cell membrane and inside the cell causing cells to swell and possibly lyse. Isotonic solutions do not alter the osmotic equilibrium across the cell membrane. Tap water and distilled water are examples of hypotonic solutions. Seawater is an example of a hypertonic solution. Medical practitioners consequently treat patients with isotonic solutions when irrigating mucosal tissues. Saline, which contains about 0.9% (w/w) sodium chloride in aqueous solution, is the most commonly used isotonic solution.

The present formulations include unique blends of multiple inorganic salts aqueous solutions at a physiologically compatible pH. Specifically, these inorganic salts include both of alkaline and alkaline earth metals at concentrations that are hypotonic relative to standard saline solution. However, these hypotonic solutions are designed to mimic the concentration of alkaline and alkaline earth metals and salts found in the endotracheal fluid of patients, which contain much lower concentrations of sodium chloride than is found in normal saline.

Native saliva and normal secretions of the trachea and nose contain about 8 mEq of sodium per liter of fluid. In contrast, 0.9% (w/w) saline contains 154 mEq sodium per liter. The use of 0.9% (w/w) saline for irrigation results in an extremely high concentration of sodium and chloride in the trachea, nose, and mouth, which inactivates the innate immunity of the airway and renders patients more vulnerable to infection.

The formulations and methods described herein may be employed to ameliorate innate host defense compared to conventional applications of 0.9% (w/w) saline in the trachea of patients. Mucosal tissues serve as an important barrier to foreign pathogens. Innate host defense includes the production of antimicrobial factors including cationic antimicrobial protein of 18 kDa (CAP 18), its C-terminal domain LL37 subunit whether conjugated or independent, other cathelicidins, lysozyme and lactoferrin. The formulations described herein do not deactivate the antimicrobial activity of native proteins or flora in innate host defense. In addition, the formulations are physiologically compatible with native tracheal mucosal secretions.

Solution constituents for synthetic, substitute, or supplemental irrigants may include one or more of the following ingredients: purified water, distilled/dionized, in an amount up to about 99.8% (w/w) and more specifically between about 95.0-100.0% (w/w) and even more specifically about 99.6997% (w/w); potassium phosphate, monobasic, anhydrous (KH₂PO₄) in an amount up to about 0.05% (w/w) and more specifically between about 0.0206-0.0251% (w/w) and even more specifically about 0.228% (w/w); and dipotassium phosphate, dibasic, anyhydrous (K₂HPO₄) in an amount up to about 0.05% (w/w) and more specifically between about 0.0217-0.0265% (w/w) and even more specifically about 0.0241% (w/w); sodium chloride (NaCl) in an amount up to about 0.1% (w/w) and more specifically between about 0.0424-0.0519% (w/w) and even more specifically about 0.0471% (w/w); potassium chloride (KCl) up to about 0.25% (w/w) and more specifically between about 0.1470-0.1797% (w/w) and even more specifically about 0.1634% (w/w); calcium chloride, dehydrate (CaCl₂.2H₂O) in an amount up to about 0.05% (w/w) and more specifically between about 0.0248-0.0303% (w/w) and even more specifically about 0.0275% (w/w); and magnesium sulfate, heptahydrate (MgSO₄.7H₂O) in an amount up to about 0.03% (w/w) and more specifically between about 0.0138-0.0168% (w/w) and even more specifically about 0.0153% (w/w). The solutions have a pH from about 5.5 to about 7.0 and more preferably from about 6.0 to about 6.5. The following table summarizes one specific example of the percentages described above.

TABLE 1

| Ingredients in Manufacturing Formulation | Formula | % (w/w) |
|---|---|---|
| Water, distilled/dionized; USP quality | H₂O | 99.6997% |
| Potassium phosphate, monobasic, anhydrous | KH₂PO₄ | 0.0228% |
| Potassium phosphate, dibasic, anyhydrous | K₂HPO₄ | 0.0241% |
| Sodium chloride | NaCl | 0.0471% |
| Potassium chloride | KCl | 0.1634% |
| Calcium chloride, dihydrate | CaCl₂•2H₂O | 0.0275% |
| Magnesium sulfate, heptahydrate | MgSO₄•7H₂O | 0.0153% |
| Totals: | | 100.0000% |

The sodium concentration as a single component or in combination with other alkaline and alkaline earth metal cations does not exceed an amount that it deactivates or significantly reduces innate host antimicrobial activity. For example, the concentration of sodium by itself or in conjunction with other alkaline and alkaline earth metal cations does not deactivate or significantly reduce antimicrobial activity of LL-37.

In some embodiments, the maximum sodium concentration is less than about 0.05% (w/w). In other embodiments, the maximum sodium concentration is less than about 0.04% (w/w). In still other embodiments, the maximum sodium concentration is less than about 0.035% (w/w). In further embodiments, the maximum sodium concentration is less than about 0.03% (w/w). In additional embodiments, the maximum sodium concentration is less than about 0.025% (w/w).

Furthermore, the concentration of a sodium salt as a single component or in combination with other alkaline and alkaline earth metal salts is not so hypotonic that chronic application of a formulation causes airway closure. The hypotonic formulations can be administered in acute applications without causing airway closure or significant airway closure.

In some embodiments, the minimum sodium concentration is greater than about 0.01% (w/w) (about 1.7 mEq/L). In other embodiments, the minimum sodium concentration is greater than about 0.03% (w/w) (about 5.1 mEq/L). In additional embodiments, the minimum sodium concentration is greater than about 0.05% (w/w) (about 8.5 mEq/L). In further embodiments, the minimum sodium concentration is greater than about 0.1% (w/w) (about 17 mEq/L). In still further embodiments, the minimum sodium concentration is greater than about 0.15% (w/w) (about 25.5 mEq/L). In still other embodiments, the minimum sodium concentration is greater than about 0.2% (w/w) (about 34 mEq/L).

Tracheal aspirates may be drawn periodically to monitor for inflammatory responses to hypotonic conditions. Inflammatory responses may indicate development of airway closure. Typically, tracheal aspirates could be drawn (1) at the time of intubation (or within 30 minutes of intubation) for information relative to congenital respiratory inflammation, (2) when an ET (endotracheal) tube is changed for any reason (within 30 minutes of placing the new tube), for information regarding tracheal colonization (as opposed to ET tube colonization), (3) when the character of the ET secretions change substantially, having become yellow, thick, and/or copious, looking for a dominant isolate with accompanying inflammatory cells, and (4) as part of the diagnostic evaluation (along with CBC, blood culture, chest X-ray, and CRP), when pneumonia is suspected in an intubated patient (to supply information relative to a dominant isolate with accompanying inflammatory cells). Culture, gram stain, and leukocyte estimation tests may be examined for indications of airway inflammation and closure.

In some embodiments, the maximum alkaline and alkaline earth metal cation concentration does not exceed an amount that deactivates or significantly reduces innate host antimicrobial activity. For example, the concentration of alkaline and alkaline earth metal cations does not deactivate or significantly reduce antimicrobial activity of LL-37.

In some embodiments, the maximum alkaline and alkaline earth metal cation concentration is less than about 0.5% (w/w). In other embodiments, the maximum alkaline and alkaline earth metal cation concentration is less than about 0.4% (w/w). In still other embodiments, the maximum alkaline and alkaline earth metal cation concentration is less than about 0.35% (w/w). In further embodiments, the maximum alkaline and alkaline earth metal cation concentration is less than about 0.3% (w/w). In additional embodiments, the maximum alkaline and alkaline earth metal cation concentration is less than about 0.25% (w/w).

Furthermore, the concentration of alkaline and alkaline earth metal cations is not so hypotonic that chronic application of a formulation causes airway closure. The hypotonic formulations can be administered in acute applications without causing airway closure or significant airway closure.

In some embodiments, the minimum alkaline and alkaline earth metal cation concentration is greater than about 0.01% (w/w). In other embodiments, the minimum alkaline and alkaline earth metal cation concentration is greater than about 0.03% (w/w). In additional embodiments, the minimum alkaline and alkaline earth metal cation concentration is greater than about 0.05% (w/w). In further embodiments, the minimum alkaline and alkaline earth metal cation concentration is greater than about 0.1% (w/w). In still further embodiments, the minimum alkaline and alkaline earth metal cation concentration is greater than about 0.15% (w/w). In still other embodiments, the minimum alkaline and alkaline earth metal cation concentration is greater than about 0.2% (w/w).

In some formulation embodiments, the alkaline and alkaline earth metal cations include sodium, potassium, calcium, and/or magnesium cations. In other formulation embodiments, the alkaline and alkaline earth metal cations include potassium, calcium, and magnesium cations. In additional formulation embodiments, the alkaline and alkaline earth metal cations include potassium and calcium cations. In still other formulation embodiments, the alkaline and alkaline earth cations include potassium and magnesium cations.

In other formulation embodiments, the alkaline and alkaline earth metal cations include sodium, calcium, and magnesium cations. In additional formulation embodiments, the alkaline and alkaline earth metal cations include sodium and calcium cations. In still other formulation embodiments, the alkaline and alkaline earth metal cations include sodium and magnesium cations.

In other formulation embodiments, the alkaline and alkaline earth metal cations include sodium, potassium, and calcium cations. In additional formulation embodiments, the alkaline and alkaline earth metal cations include sodium, potassium, and magnesium cations.

In other formulation embodiments, the alkaline and alkaline earth metal cations include alkaline cations such as sodium by itself, potassium by itself or sodium and potassium. In additional formulation embodiments, the alkaline and alkaline earth metal cations include alkaline earth metal cations such as calcium by itself, magnesium by itself, or calcium and magnesium.

In embodiments where alkaline metal cations are present, the concentration of alkaline metal cations does not exceed an amount that it deactivates or significantly reduces innate host antimicrobial activity. For example, the total concentration of sodium and potassium does not deactivate or significantly reduce antimicrobial activity of LL-37.

In some embodiments, the maximum sodium concentration is less than about 0.5% (w/w) (about 85 mEq/L). In other embodiments, the maximum sodium concentration is less than about 0.4% (w/w) (about 68 mEq/L). In still other embodiments, the maximum sodium concentration is less than about 0.35% (w/w) (about 60 mEq/L). In further embodiments, the maximum sodium concentration is less than about 0.3% (w/w) (about 51 mEq/L). In additional embodiments, the maximum sodium concentration is less than about 0.25% (w/w) (about 43 mEq/L).

Furthermore, the concentration of alkaline metal is not so hypotonic that chronic application of a formulation causes airway closure. The hypotonic formulations can be administered in acute applications without causing airway closure or significant airway closure.

In some embodiments, the minimum alkaline metal cation concentration is greater than about 0.01% (w/w) (about 1.7 mEq/L). In other embodiments, the minimum alkaline metal cation concentration is greater than about 0.03% (w/w) (about 5.1 mEq/L). In additional embodiments, the minimum alkaline metal cation concentration is greater than about 0.05% (w/w) (about 8.5 mEq/L). In further embodiments, the minimum alkaline metal cation concentration is greater than about 0.1% (w/w) (about 17 mEq/L). In still further embodiments, the minimum alkaline metal cation concentration is greater than about 0.15% (w/w) (about 25.5 mEq/L). In still other embodiments, the minimum alkaline metal cation concentration is greater than about 0.2% (w/w) (about 34 mEq/L).

In some embodiments, the solution may be used a relatively short time after development, for example, several hours. In other embodiments, the solution may be stored for a period of time, for example about 4-6 months at room temperature or other atmospheric condition, or at various locations. In certain embodiments, a problem may occur where small amounts of precipitate or crystals from compounds or molecules form during the preparation stage or after the solution has been stored for a period of time. Additionally, the temperature or method of mixing may influence whether or not these precipitates form, or increase the likelihood that they form within the solution. Even if these deposits may not be dangerous for consumption, their observation may be undesirable for consumer perception of the solution.

The identification of these particulates may be performed by collecting the solids and separating them from their respective solutions. These solids are dissolved in nitric acid and analyzed for elemental composition. In some embodiments, the particulates may be salts of calcium and phosphate, with varying ratios. The following table illustrates one example of the approximate concentrations in ppm for each element in a resulting solution after being placed in storage for 4-6 months.

TABLE 2

| # | C | Na | Mg | P | Cl | K | Ca | Ca/P |
|---|---|---|---|---|---|---|---|---|
| 1 | 3 | 4 | 1 | 47 | 9 | 1 | 102 | 2.2 |
| 2 | 3 | 3 | 0 | 49 | 11 | 2 | 83 | 1.7 |
| 3 | 3 | 2 | 0 | 8 | 8 | 0 | 19 | |
| 4 | 3 | 2 | 0 | 8 | 10 | 1 | 21 | |
| 5 | 7 | S | 15 | 72 | 2265 | 20 | 66 | |
| 6 | 8 | S | 13 | 60 | 2279 | 20 | 41 | |
| 7 | 4 | S | 13 | 56 | 2395 | 20 | 37 | |
| 8 | 3 | 6 | 3 | 123 | 30 | 8 | 255 | 2.1 |
| 9 | 6 | S | 12 | 60 | 2232 | 21 | 59 | |
| 10 | 4 | S | 11 | 50 | 1870 | 21 | 30 | |
| 11 | 2 | 3 | 0 | 200 | 28 | 8 | 300 | 1.5 |
| 12 | 4 | S | 13 | 54 | 1883 | 21 | 37 | |
| 13 | 3 | 4 | 0 | 312 | 39 | 12 | 462 | 1.5 |
| 14 | 5 | S | 14 | 53 | 2097 | 22 | 33 | |
| 15 | 4 | S | 14 | 62 | 2122 | 22 | 45 | |
| 16 | 5 | S | 12 | 55 | 2064 | 22 | 42 | |
| Control | 2 | 0 | 0 | 0 | 4 | 0 | 0 | |
| dilute | | | | | | | | |
| H⁺ | | | | | | | | |
| Control | 4 | 2 | 0 | 0 | 8 | 0 | 0 | |
| conc H⁺ | | | | | | | | |

In Table 2, samples 1, 2, 8, 11, and 13 represent recovered solids re-dissolved in nitric acid. The other samples were liquids or controls for comparison.

Calcium phosphate is very insoluble in water, and can cause cloudiness and precipitates similar to those observed in the samples of the formulation. In determining the effects of calcium and phosphate concentrations on precipitate formation, multiple series of solutions were prepared with varying concentrations of calcium and phosphate compounds. For example, several solutions with varying amounts of mono-basic and di-basic forms of phosphate were varied to simultaneously test their effects and associated pH changes as the main buffering components in the formulation. Additionally, different ranges of heat were applied to the solutions at different stages of preparation, for example, high temperatures from 60-94° C., room temperature ranging from 25-37° C., cold temperatures above 0° C. and freezing temperatures, for example −20° C. Other ranges could easily be tested.

Results and statistical evaluation of these formulation trials and physical testing show that calcium and phosphates were among contributing factors to the formation of precipitates. Additionally, the ratio of mono-basic to di-basic phosphates did not create a substantial difference in total precipitates formed, across the pH ranges tested.

The foregoing represents one test that was done to determine the cause of precipitate formation. Other series of tests may be performed with varying concentrations of calcium and phosphate compounds, for example, calcium chloride and potassium phosphates. In performing these tests, formation of precipitates decreased substantially when the concentration of calcium chloride was decreased. In addition, the formation of precipitates decreased when mono-basic and di-basic phosphates were decreased.

In one embodiment, solution constituents for synthetic, substitute, or supplemental irrigants may include one or more of the following ingredients: purified water, distilled/dionized, in an amount up to about 99.8% (w/w) and more specifically between about 95.0-100.0% (w/w) and even more specifically about 99.6998% (w/w); potassium phosphate, monobasic, anhydrous (KH₂PO₄) in an amount up to about 0.05% (w/w) and more specifically up to about 0.0300% (w/w) and even more specifically about 0.010% (w/w), or as low as about 0.005% (w/w) or 0.001% (w/w); and dipotassium phosphate, dibasic, anyhydrous (K₂HPO₄) in an amount up to about 0.05% (w/w) and more specifically up to about 0.0300% (w/w) and even more specifically about 0.0100% (w/w), or as low as 0.001% (w/w); sodium chloride (NaCl) in an amount up to about 0.1% (w/w) and more specifically between about 0.0424-0.0519% (w/w) and even more specifically about 0.0471% (w/w); potassium chloride (KCl) up to about 0.30% (w/w) and more specifically between about 0.10-0.30% (w/w) more specifically between about 0.1470-0.2200% (w/w) and even more specifically about 0.2134% (w/w); calcium chloride, dehydrate (CaCl₂.2H₂O) in an amount up to about 0.05% (w/w) and more specifically up to about 0.0303% (w/w) and even more specifically about 0.0015% (w/w), and in another embodiment between about 0.0005-0.0300% (w/w); and magnesium sulfate, heptahydrate (MgSO₄.7H₂O) in an amount up to about 0.030% (w/w) and more specifically between about 0.0138-0.0212% (w/w) and even more specifically about 0.0182% (w/w), or as low as about 0.005% (w/w). The solutions have a pH from about 5.5 to about 7.0 and more preferably from about 6.0 to about 6.5. The following tables summarize a number of specific examples of the ranges of percentages described above.

TABLE 3

| | Water | KH₂PO₄ | K₂HPO₄ | NaCl | KCl | CaCl₂•2H₂O |
|---|---|---|---|---|---|---|
| 1 | 99.6998% | 0.0100% | 0.0100% | 0.0471% | 0.2134% | 0.0015% |
| 2 | 99.6998% | 0.0300% | 0.0100% | 0.0471% | 0.2134% | 0.0015% |
| 3 | 99.6998% | 0.0100% | 0.0300% | 0.0471% | 0.2134% | 0.0015% |
| 4 | 99.6998% | 0.0300% | 0.0300% | 0.0471% | 0.2134% | 0.0015% |
| 5 | 99.6998% | 0.0100% | 0.0100% | 0.0471% | 0.2134% | 0.0030% |
| 6 | 99.6998% | 0.0300% | 0.0100% | 0.0471% | 0.2134% | 0.0030% |
| 7 | 99.6998% | 0.0100% | 0.0300% | 0.0471% | 0.2134% | 0.0030% |
| 8 | 99.6998% | 0.0300% | 0.0300% | 0.0471% | 0.2134% | 0.0030% |
| 9 | 99.6998% | 0.0200% | 0.0200% | 0.0471% | 0.2134% | 0.0022% |

TABLE 4

| | | | | | | | Avg |
|---|---|---|---|---|---|---|---|
| | MgSO₄•7H₂O | Total | ppt | pH 1 | pH 2 | pH 3 | pH |
| 1 | 0.0182% | 100.0000% | None | 6.44 | 6.39 | | 6.42 |
| 2 | 0.0182% | 100.0200% | None | 5.98 | 5.93 | | 5.96 |
| 3 | 0.0182% | 100.0200% | None | 6.87 | 6.85 | | 6.86 |
| 4 | 0.0182% | 100.0400% | None | 6.41 | 6.39 | | 6.40 |
| 5 | 0.0182% | 100.0015% | ppt | 6.48 | 6.40 | | 6.44 |
| 6 | 0.0182% | 100.0215% | ppt | 5.97 | 5.94 | | 5.96 |
| 7 | 0.0182% | 100.0215% | None | 6.84 | 6.86 | | 6.85 |
| 8 | 0.0182% | 100.0415% | None | 6.42 | 6.41 | | 6.42 |
| 9 | 0.0182% | 100.0207% | None | 6.48 | 6.43 | 6.40 | 6.44 |

From the above described experiments, it was determined that pH in the range of about 5-8 is not a primary cause of precipitation, with even lower pH values showing slight ppt. Calcium is a substantial cause of precipitation, and when decreased to <0.0020%, no ppt is observed. The amount of calcium within the solution may be minimized by preparing the formula from distilled or deionized water. Additionally, phosphate is a substantial cause of precipitation, and when decreased to 0.01% with a lower amount of calcium, no ppt is observed. Additionally, when both the H₂PO₄⁻ and HPO₄²⁻ species are decreased, the pH is decreased from about 6.61 to about 6.42. Additionally, because calcium concentration is decreased, the concentration of magnesium may be increased by increasing amounts of magnesium sulfate in the formulation. Furthermore, to keep the total dissolved solids up to about 0.30%, the level of potassium chloride may be increased. The following table summarizes one specific example of the percentages described above.

TABLE 5

| Ingredients in Manufacturing Formulation | Formula | % (w/w) |
|---|---|---|
| Water, distilled/dionized; USP quality | H₂O | 99.6998% |
| Potassium phosphate, monobasic, anhydrous | KH₂PO₄ | 0.010% |
| Potassium phosphate, dibasic, anyhydrous | K₂HPO₄ | 0.010% |
| Sodium chloride | NaCl | 0.0471% |
| Potassium chloride | KCl | 0.2134% |
| Calcium chloride, dihydrate | CaCl₂•2H₂O | 0.0015% |
| Magnesium sulfate, heptahydrate | MgSO₄•7H₂O | 0.0182% |
| Totals: | | 100.0000% |

The embodiment of the formulation in table 5 was prepared under various conditions and then tested. The tests and preparations applied to this embodiment may be applied to other embodiments as well.

In one embodiment, the formula was prepared by first heating the water to 94° C., then mixing the remaining ingredients in order from top to bottom of table 5. In another embodiment, the same solution was cooled and frozen overnight at 20° C., and thawed for inspection. In another embodiment, another solution was prepared at room temperature and heated to 60° C., then subsequently cooled to room temperature. In another embodiment, the solution that had been heated to 60° C. was frozen for a period of time, for example, one day, then thawed for inspection. In other embodiments, the amount of each ingredient was increased and decreased by 10% of the indicated amount on table 5. In another embodiment, the pH was altered by varying the amounts of KH₂PO₄ and K₂HPO₄ within about 10% of the values in table 5, and prepared for inspection. Many of these embodiments were tested alone and in combination with other embodiments to test for threshold values in determining whether precipitates would form. In each of the embodiments, precipitates and cloudiness were not observed.

Optionally, the formulations may also contain one or more therapeutically active agents. Such therapeutically active agents may include over-the-counter and prescription drugs, antifungal agents, antiviral agents, antibacterial agents, corticosteroids, lysozme, saliva production agents, oral antiseptics, anticariogenic agents, oral mucosa protective agents, and the like.

Solution constituents for substitute or supplemental irrigants could also be expressed in terms of total osmolarity of from about 10 to about 660 milliosmoles/L, from about 64 to about 461 milliosmoles/L, and from about 164 to about 381 milliosmoles/L.

In another aspect, the irrigant formulations have a total ionic osmolarity of less than about 154 milliosmoles/L. In some embodiments, the total ionic osmolarity is less than about 120 milliosmoles/L. In additional embodiments, the total ionic osmolarity is less than about 100 milliosmoles/L. In other embodiments, the total ionic osmolarity is less than about 85 milliosmoles/L. In still other embodiments, the total ionic osmolarity is less than about 80 milliosmoles. In even other embodiments, the total ionic osmolality is less than about 75 milliosmoles/L. In yet other embodiments, the total ionic osmolarity is less than about 70 milliosmoles/L.

These formulations can provide both acute and long term relief and act as lubricants for patients receiving ventilation assistance. The formulations may be swallowed to provide relief to the upper digestive track, including the esophagus. The formulations may also be applied through the nasal and sinus cavities.

The formulations may also be used to prevent or wash away mucosal build ups including tracheal build ups, which may partially or completely occlude the lumen of respiratory support equipment (thus washing open an occlusion). The formulations provide an aqueous mucosal tissue irrigant with sufficient lubricity to prevent, wash away, or wash open mucosal occlusion in and around respiratory equipment without deactivating the antimicrobial activity of innate host defense.

Optionally, one or more oral antiseptics such as triclosan may be included for their antiseptic activity against pathogenic microorganisms. The irrigant formulations can include between 0.01 and 2.0% (w/w) and preferably between 0.1 and 0.3% (w/w) antiseptic, with respect to the total weight/volume.

Optionally, one or more anticariogenic agents such as fluoride compounds, including sodium fluoride (NaF) and sodium monofluorophosphate, also known as NaMFP, may be included. The irrigant formulations can include between 0.01% (w/w) and 2.0% (w/w) and preferably between 0.1 and 0.3% (w/w) anticariogenic agents, with respect to the total weight/volume. Xylitol also can act as an anticariogenic agent to inhibit the growth of certain bacteria present in the mouth and also help reduce demineralization of tooth enamel.

Optionally, one or more protective agents for the oral mucosa, particularly the gingival mucosa may be included. These protective agents include vitamin E acetate and the like to improve blood circulation and inhibit inflammation.

Panthenol is a pantothenic acid derivative that promotes the healing process by epidermal stimulation without causing sensitization or irritation. The extracts of Aloe Vera are natural products of proven efficacy that inhibit inflammation and improving the healing of wounds. The irrigant formulations may include between 0.01% (w/w) and 1.0% (w/w) and preferably between 0.1 and 0.3% (w/w) mucosa protective agents, with respect to the total weight/volume.

The irrigants may serve as irrigant formulations and can further include water and/or polyols acceptable for human consumption, among them glycerin, sorbitol, and/or propylene glycol, as well as with other nonessential ingredients of those conventionally used in oral liquid formulations.

In another aspect, the formulations may also include a surfactant. In some embodiments, the surfactant is a respiratory surfactant. Surfactants may be used to affect the surface tension of a fluid. Surface tension is the tendency of molecules in a fluid to be pulled toward the center of the fluid. Examples of surfactants include but are not limited to phospholipids, proteins, and cholesterols.

The surfactant may be ionic or non-ionic. Protein surfactants may be surfactant-associated proteins (such as SP-A to SP-D). The surfactants may be synthetic or natural. Natural surfactants are derived from animal sources. Synthetic surfactants can include complex combinations of phospholipids, neutral lipids, lipid proteins or alcohols.

Examples for artificial surfactants are Exosurf Neonatal (a synthetic surfactant composed of dipalmitoylphosphatidylcholine, hexadecanol, and tyloxapol), Lucinactant (Surfaxin®, Discovery Laboratories, Inc., Warrington, Pathologic., USA) (a mixture of dipalmitoylphosphatidylcholin-e, palmitoyloleoylphosphatidylglycerol, palmitic acid, and KL4) or Lusupultide (Venticute©, Altana Pharma, Konstanz, Germany) (a mixture of phospholipids and recombinant SP-C). Exemplary natural surfactants include Curosurf®, Survanta©, or Alveofact®, however, other preparations of surfactant could be used equally. Curosurf® (Chiesi company, Italy) is a lipid extract from whole minced porcine lung tissue. Survanta® (Abbott GmbH, Wiesbaden, Germany) is prepared from minced bovine lung extract with added DPPC, triacylglycerol, and palmitic acid. Alveofact® (Boehringer Ingelheim Pharma KG, Ingelheim, Germany) is produced by lipid extraction from bovine lung lavage.

In yet another aspect, the formulations may also include an optional flavoring agent. The flavoring agent may be used to affect the taste of the irrigant solution.

The formulations may be delivered in a variety of manners including with “bullet tubes” as previously practiced or by volumetric syringes, metered dose devices, droppers or other devices. A specific example of a storage container and delivery device is disclosed in U.S. Provisional Patent Application No. 61/394,719, entitled “Ampoule with protective sleeve for contamination prevention,” which was filed on Oct. 19, 2010, and is incorporated by reference herein. As mentioned earlier, the formulations contemplated may be presented in a variety of forms including mouthwashes, sprays and oral gels.

In addition to water, polyhydroxylated compounds such as glycerin or glycols (e.g., propylene glycol, nonionic surfactants, etc.) and other additives to improve appearance, flavor, and preservation can be included. For embodiments which do not include preservatives, care may be taken to sterilize the solution before, during, or after a filling process to put the solution into a storage container. Sterilization procedures may help to insure that the resulting solution is non-pyrogenic. As one example, the solution may be gamma sterilized once it is stored in plastic ampoules. In another example, the filling process may be aseptic.

The sprays are formulations equal or similar to mouthwashes but dispensed in spray bottles for convenient application of the dose needed to moisten and protect the trachea without requiring subsequent rinsing.

Oral gel formulations of the substitute saliva can include polymers that impart gel qualities and texture to the formulations. Oral gels may be administered by direct application to the oral cavity. Such polymers include polycarbophil and carbomer, since they keep the gel structure stable for very prolonged times under extreme temperature conditions.

Some embodiments of the formulations are prepared by conventional mixing techniques. In a more specific embodiment, the formulation is prepared according to strict procedures in order to avoid the formation of precipitates. In one embodiment, the ingredients are mixed according to the following procedures:
1. Charge a tank (e.g., Teflon-lines metal or stainless steel) with distilled/deionized water of USP quality;
2. Add potassium phosphate and dipotassium phosphate to the water with stirring/agitation to insure complete dissolution;
3. Add sodium chloride, potassium chloride, calcium chloride, and magnesium sulfate to the water mixture with stirring/agitation to insure complete dissolution;
4. Store the tank and mixture at about 2-8° C. to inhibit microbial growth (when not immediately drawing material for filling ampoules);
5. Keep the tank and mixture covered and sealed during storage.

In other embodiments, the formulation may be manufactured with different steps and/or in a different order. For example, the potassium phosphate and diposssium phosphate may be added to a smaller quantity of water, and the remaining ingredients may be added to another quantity of water, and then the two mixtures subsequently may be mixed together into a single solution. Additionally, the final solution may be heated to a high temperature (e.g., above about 90° C.) to help eliminate microbial growth or kill microorganisms that might be introduced during the manufacturing process.

In manufacturing the formulation, hot or cold water may be used during the compounding of the formula. Additionally, the formulation may be prepared in a large container for economical purposes as well as preventing potential cloudiness and formation of precipitates in the formula.

In some embodiments, the solution may be prepared by first heating the water, then manufacturing the formulation with different steps and/or a different order. The solution may also be heated or frozen prior to administration. In some embodiments, the solution may be prepared at room temperature and neither heated nor frozen prior to administration.

The formulations described may be administered to a patient's trachea and, in particular, to infants to reduce infections including iatrogenic nosocomial infections. Therefore, a method for providing an oral lubricant to a patient includes orally administering an effective amount of a liquid formulation of the ingredients described herein. When desired, the formulations may be swallowed or expectorated. When swallowed or otherwise advanced to the gastrointestinal tract, the formulations may be used to lavage the gastrointestinal tract. The formulations may be administered at periodic intervals.

In some embodiments, the methods of irrigation can include contacting a mucosal tissue with any of the formulations with alkaline and alkaline earth metal cations and concentration ranges described above.

In additional embodiments, the methods of irrigation can include contacting a mucosal tissue with a solution with a lower ionic strength than conventional irrigants. By irrigating mucosal areas with lower ionic strength fluids, endogenous antimicrobials maintain antimicrobial activity to impart innate host defense.

In other embodiments, the methods can include contacting a mucosal tissue with a solution with a lower sodium chloride concentration than conventional irrigants. By irrigating mucosal areas with lower sodium chloride concentration fluids, endogenous antimicrobials maintain activity in contributing to innate host defense.

In still other embodiments, the methods can include contact a mucosal tissue with a solution with low levels of sodium chloride and alkaline earth metal salts. By irrigating mucosal areas with lower sodium chloride concentration and alkaline earth metal salts, endogenous antimicrobials maintain or improve activity in contributing to innate host defense.

Caregivers can irrigate and lubricate the endotracheal tube or other similar conduits as well as the airway in patients who require mechanical ventilation. The methods and formulations described can prevent tracheal and other similar secretions from building up and occluding the lumens of the endotracheal tube and airway. Furthermore, the methods and formulations described prevent tracheal abrasion from respiratory equipment, which may further irritate or inflame both mucosal and adjoining tissues. Applying the formulations can reduce the ionic concentration of sodium chloride and other salts, which grows as a result of evaporation, and the lack of lip apposition. Furthermore, the methods and formulations described herein avoid the problems associated with iatrogenically adding excessive sodium and/or alkaline and alkaline earth metal cations that deactivate innate host defense. The methods described deliver water to mucosal surfaces at physiologically salt acceptable concentrations while at the same time providing both an irrigating and lubricating function.

It is contemplated that the irrigant formulations described herein can be used to irrigate or lubricate a variety of tissues and areas of an organism's body. These tissues and organisms include, but are not limited to the mouth, lips, teeth, gums, tongue, sinus passages, lungs, esophagus, throat, nose, Eustachian tubes, ears, eyes, larynx, and the like.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A synthetic endotracheal irrigant comprising:
a water content of up to about 98% (w/w);
a total mineral content of less than about 5.0% (w/w), including a sodium content of less than about 2.0% (w/w);
wherein the synthetic endotracheal irrigant is exclusive of protein.

2. A synthetic endotracheal irrigant which substantially mimics natural endotracheal fluids in terms of mineral content and fluid characteristics, but which excludes protein.

3. The synthetic endotracheal irrigant of claim 2, further consisting essentially of:
water in a relative quantity of about 95.0% (w/w) to about 99.8% (w/w);
potassium phosphate, monobasic, anhydrous in a relative quantity of about 0.001% (w/w) to about 0.04% (w/w);
dipotassium phosphate, dibasic, anyhydrous in a relative quantity of about 0.001% (w/w) to about 0.04% (w/w);
sodium chloride in a relative quantity of about 0.03% (w/w) to about 0.06% (w/w);
potassium chloride in a relative quantity of about 0.10% (w/w) to about 0.30% (w/w);
calcium chloride, dihydrate in a relative quantity of about 0.0005% (w/w) to about 0.0300(w/w); and
magnesium sulfate, heptahydrate in a relative quantity of about 0.005% (w/w) to about 0.030% (w/w);
wherein the percentages are weight-weight percentages.

4. The synthetic endotracheal irrigant of claim 3, further consisting essentially of:
water in a relative quantity of about 99.6998% (w/w);
potassium phosphate, monobasic, anhydrous in a relative quantity of about 0.010% (w/w);
dipotassium phosphate, dibasic, anyhydrous in a relative quantity of about 0.010% (w/w);
sodium chloride in a relative quantity of about 0.0471% (w/w);
potassium chloride in a relative quantity of about 0.2134% (w/w);
calcium chloride, dihydrate in a relative quantity of about 0.0015% (w/w); and
magnesium sulfate, heptahydrate in a relative quantity of about 0.0182% (w/w).

5. A method of making a synthetic endotracheal irrigant, wherein the method comprises:
adding water to a mixing container;
adding potassium phosphate and dipotassium phosphate to the water;
substantially dissolving the potassium phosphate and the dipotassium phosphate into the water;
adding sodium chloride, potassium chloride, calcium chloride, and magnesium sulfate to the water containing the dissolved potassium phosphate and the dipotassium phosphate; and
substantially dissolving the sodium chloride, potassium chloride, calcium chloride, and magnesium sulfate into the water containing the dissolved potassium phosphate and the dipotassium phosphate.
